(19) United States
(12) Patent Application Publication
(10) Pub. No.: US 2002/0042653 A1
(43) Pub. Date: Apr. 11, 2002

(54) Blue blocking intraocular lens implant

(76) Inventors: Victor L. Copeland, Rancho Santa Fe, CA (US); Steven G. Pratt, Del Mar, CA (US)

Correspondence Address: STETINA BRUNDA GARRED & BRUCKER, 75 ENTERPRISE, SUITE 250, ALISO VIEJO, CA, 92656, US

(21) Appl. No.: 10/014,298

(22) Filed: Dec. 11, 2001

Related U.S. Application Data
(63) Continuation of application No. 09/448,194, filed on Nov. 23, 1999.

(51) Int. Cl. A61F 2/16

(52) U.S. Cl. 623/6.6; 623/905; 623/6.62

(57) ABSTRACT

A surgically implantable synthetic intraocular lens having a wavelength blocker for blocking transmission of 100% or at least a portion of blue light of wavelengths between about 400 and 510 nanometers, as well as for blocking ultraviolet A and B wavelengths and optionally infrared wavelength transmissions. A preferred wavelength transmission blocker is a tint composition either disposed exteriorly upon the lens or blended interiorly within the lens. Lens utility is found in replacement of the natural crystalline lens to achieve visual correction of aphakia, as well as in correcting refractive errors of the eye through implantation without removal of the natural crystalline lens to thereby provide filtering properties that yield maximum visual acuity along with blockage of untoward wavelength transmissions. The lens implants thereby support vision health by protecting eyes from damage caused through the transmission of untoward wavelengths to the interior of the eye.

### FIELD OF THE INVENTION

The present invention relates in general to synthetic lens construction for intraocular lens implants that replace the crystalline lens and intraocular lenses that correct refractive error, and in particular to such lenses having a wavelength filter for preventing transmission of 100% or lesser amounts of violet and blue light between about 400 and 510 nanometers, and in conjunction with blocking 100% of ultraviolet A and B wavelengths and optionally filtering infrared wavelength transmissions.

### BACKGROUND OF THE INVENTION

The incidence of cataracts and macular degeneration is increasing worldwide, with the causes thereof ranging from atmospheric ozone depletion which allows increased damaging electromagnetic radiation, to violet and blue light wavelengths, ultraviolet A and B wavelengths, and infrared wavelengths which damage vulnerable eye components. A specific potentially dangerous situation occurs where a synthetic intraocular lens implant is surgically placed in the eye of a patient after removal of the crystalline lens. In particular, as the human lens ages it is subject to nuclear sclerosis, known as “brunescent cataract,” which, because of its color, blocks at least a portion of blue light entry generally up to about 450 nanometers. In fact, human studies show that a normal 53-year-old crystalline lens transmits only 10-50% of visible blue light to the retina. Because this blue light can be quite damaging to the retina, such natural blockage is highly desired. If this natural lens becomes generally inoperative because of critical cataract growth, it typically is surgically replaced with a lens implant. Present lenses employed for implant, however, do not block wavelengths above 400 nanometers, thus resulting in 100% passage of blue light (400 nm to 510 nm) to the retina and resulting retina exposure to potential hazard.

In view of the retinal hazard caused by wavelength transmission through such an implanted lens, it is apparent that a need is present for intraocular protection against damaging blue light hazards, with such protection extending to blockage of ultraviolet and infrared wavelength transmissions as advisable. Accordingly, the primary object of the present invention is to provide an implantable synthetic replacement lens wherein such lens blocks at least a portion or 100% of the wavelength transmission between about 400 and 510 nanometers.

Another object of the present invention is to provide an implantable synthetic replacement lens wherein, in addition to blocking 100% or a portion thereof of visible wavelength transmission between about 400 and 510 nanometers, the lens blocks ultraviolet A and B and infrared wavelength transmissions.

Yet another object of the present invention is to provide methodology whereby a patient is provided with protection from ocularly damaging wavelength transmission through implantation of a synthetic intraocular refractive lens.

These and other objects of the invention will become apparent throughout the description thereof which now follows.

### SUMMARY OF THE INVENTION

The present invention comprises a surgically implantable synthetic intraocular lens having a wavelength blocker for blocking transmission of 100% or at least a portion of violet/blue light of wavelengths between about 400 and 510 nanometers, as well as for 100% blocking of ultraviolet A and B wavelengths and/or infrared wavelength transmissions. The preferred wavelength transmission blocker is a tint either disposed exteriorly upon the synthetic lens or blended interiorly within the synthetic lens. The magnitude of specific wavelength blockage can be varied as indicated by the needs of respective patients. Thus, the wavelength blocker of the lens can be chosen to block from 0% to 100% transmission of one or more selected wavelengths between about 400 and 510 nanometers. In this manner, a physician can permit some blue light transmission to thereby retain color perception capabilities for certain patients while blocking all blue light transmission in patients where such total blockage protection is indicated. Lens utility is found in replacement of the natural crystalline lens to achieve visual correction of aphakia, as well as in correcting refractive errors of the eye through implantation without removal of the natural crystalline lens to thereby provide filtering properties that yield maximum visual acuity along with blockage of untoward wavelength transmissions.

Lenses constructed according to the invention are surgically implantable using traditional procedures and techniques commonly employed by ophthalmic surgeons in accomplishing standard lens implantation. Thus, if a natural crystalline lens is to be replaced, the surgeon first removes the natural lens and thereafter places the wavelength-blocking intraocular lens above described. Conversely, if a natural crystalline lens is to be retained in concert with an implanted lens for correcting refractive errors, traditional implantation again is employed as commonly practiced by an ophthalmic surgeon. As is apparent, the present invention provides lens implants that support vision health by protecting eyes from damage caused through the transmission of untoward wavelengths to the retinal interior of the eye.

### BRIEF DESCRIPTION OF THE DRAWINGS

An illustrative and presently preferred embodiment of the invention is shown in the accompanying drawings in which:

FIG. 1 is an enlarged side elevational view of a surgically implantable synthetic intraocular lens.

### DETAILED DESCRIPTION OF THE PREFERRED EMBODIMENT

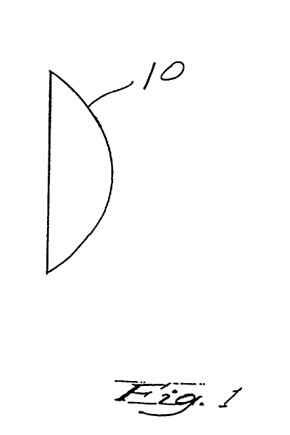

Referring to FIG. 1, a surgically implantable synthetic intraocular lens 10 is shown. The lens 10 is surgically implanted as known in the art after removal of a diseased lens, or cataract, from the eye of a patient. Conversely, if the lens 10 is to be implanted without removal of a natural lens as indicated for correcting refractive errors, the lens 10, with necessary corrective attributes, is likewise implanted as known in the art. As earlier discussed, and in addition to dangers associated with ultraviolet A, ultraviolet B, and infrared wavelengths, visible blue light (i.e. wavelengths above 400 nanometers) is now known to be potentially very dangerous to the retina component of an eye. Consequently, and in addition to blocking ultraviolet A and ultraviolet B wavelengths as well as infrared wavelengths, the lens 10 here illustrated blocks transmission there through of 100% or at least a portion of wavelengths between about 400 and 510 nanometers. Transmission prohibition is accomplished by a wavelength transmission blocker preferably being a tint composition provided as known in the art either by individual exterior tint application procedures to each lens after lens formation or by interiorly incorporating the tint composition within a block of material from which a lens is subsequently derived. Non-limiting tint composition products include those available from Brain Power, Inc., Miami, Fla., and from Phantom Tinting Co., San Diego, Calif. The lens 10 is constructed of biocompatible material, and typically will be injection molded or stamped from sheet stock and molded for direct placement into the eye. As would be apparent, should a physician not desire an implantable lens to include wavelength blockage other than at least a portion of visible blue between about 400 and 510 nanometers, a tint composition can be employed that only blocks such visible blue wavelength transmission. The lens 10 is implanted through a conventional surgical procedure, and standard recovery time for the patient is experienced while retina well-being is thereby maintained.

Because total blue light blockage between 400 and 510 nanometers causes color distortion which, in turn, could: create untoward situations such as an inability to properly distinguish traffic device colors while driving, it is preferred that less than 100% blue light blockage occurs unless absolutely necessary for patient treatment. Instead, it is preferred that at least a portion of one or more of the blue light wavelengths between about 400 and 510 nanometers be transmitted. Such transmission can be chosen to include a graduated percentage of various wavelength transmissions (e.g. 5% at 410 nanometers, 30% at 430 nanometers, etc.), a constant percentage (e.g. 30%) of some or all wavelengths between 400 and 510 nanometers, from 0% to 100% of one or more wavelengths between 400 and 510 nanometers, or the like.

While an illustrative and presently preferred embodiment of the invention has been described in detail herein, it is to be understood that the inventive concepts may be otherwise variously embodied and employed and that the appended claims are intended to be construed to include such variations except insofar as limited by the prior art.

What is claimed is:

1. A surgically implantable synthetic intraocular lens having a wavelength transmission blocker for blocking 100% or at least a portion of at least one visible wavelength between about 400 and 510 nanometers.

2. A surgically implantable synthetic intraocular lens as claimed in claim 1 wherein the wavelength transmission blocker additionally blocks wavelength transmission of wavelengths chosen from the group consisting of ultraviolet A wavelengths, ultraviolet B wavelengths, infrared wavelengths, and combinations thereof.

3. A surgically implantable synthetic intraocular lens as claimed in claim 1 wherein the wavelength transmission blocker is a tint composition.

4. A surgically implantable synthetic intraocular lens as claimed in claim 3 wherein the tint composition is disposed exteriorly upon the lens.

5. A surgically implantable synthetic intraocular lens as claimed in claim 3 wherein the tint composition is disposed interiorly within the lens.

6. A method of protecting a retina of an eye upon replacement of a diseased natural crystalline lens, the method comprising:
a) surgically removing the natural crystalline lens; and
b) implanting in place of the natural crystalline lens a synthetic intraocular lens having a wavelength transmission blocker for blocking 100% or at least a portion of at least one visible wavelength between about 400 and 510 nanometers.

7. A method of protecting a retina of an eye as claimed in claim 6 wherein the wavelength transmission blocker of the synthetic intraocular lens additionally blocks wavelength transmission of wavelengths chosen from the group consisting of ultraviolet A wavelengths, ultraviolet B wavelengths, infrared wavelengths, and combinations thereof.

8. A method of protecting a retina of an eye as claimed in claim 6 wherein the wavelength transmission blocker of the synthetic intraocular lens is a tint composition.

9. A method of protecting a retina of an eye as claimed in claim 8 wherein the tint composition is disposed exteriorly upon the lens.

10. A method of protecting a retina of an eye as claimed in claim 8 wherein the tint composition is disposed interiorly within the lens.

11. A method of correcting refractive errors of an eye while maintaining in place a natural crystalline lens of the eye, the method comprising implanting a corrective synthetic intraocular lens having a wavelength transmission blocker for blocking 100% or at least a portion of at least one visible wavelength between about 400 and 510 nanometers.

12. A method of correcting refractive errors of an eye as claimed in claim 11 wherein the wavelength transmission blocker of the synthetic intraocular lens additionally blocks wavelength transmission of wavelengths chosen from the group consisting of ultraviolet A wavelengths, ultraviolet B wavelengths, infrared wavelengths, and combinations thereof.

13. A method of correcting refractive errors of an eye as claimed in claim 11 wherein the wavelength transmission blocker of the synthetic intraocular lens is a tint composition.

14. A method of correcting refractive errors of an eye as claimed in claim 13 wherein the tint composition is disposed exteriorly upon the lens.

15. A method of correcting refractive errors of an eye as claimed in claim 13 wherein the tint composition is disposed interiorly within the lens.
